(19) United States
(12) Patent Application Publication
(10) Pub. No.: US 2026/0034161 A1
(43) Pub. Date: Feb. 5, 2026

(54) 2-HYDROXYPROPYL-BETA-CYCLODEXTRIN AS A CANCER THERAPY NEOADJUVANT OR ADJUVANT

(71) Applicant: University of the Witwatersrand, Johannesburg, Johannesburg (ZA)

(72) Inventors: Mandeep Kaur, Johannesburg (ZA); Naaziyah Abdulla, Johannesburg (ZA); Ruth Aronson, Johannesburg (ZA); Shanen Perumal; Ruvesh Pillay, Johannesburg (ZA)

(21) Appl. No.: 18/996,685

(22) PCT Filed: Jul. 20, 2023
(86) PCT No.: PCT/IB2023/057400

§ 371 Date: Jan. 17, 2025

(30) Foreign Application Priority Data
Jul. 20, 2022 (ZA) 2022/08061

(51) Int. Cl. A61K 31/724; A61K 45/06; A61P 35/00

(52) U.S. Cl. CPC A61K 31/724; A61K 45/06; A61P 35/00

(57) ABSTRACT

The invention relates to a compound, 2-hydroxypropyl-β-cyclodextrin (HPβCD), for use in methods of treating cancer, as well as to methods of treating a cancer using the compound HPβCD and to uses of the compound in the manufacture of one or more medicaments for the treatment of cancer. In particular, the compound HPβCD may be useful as an adjuvant or neoadjuvant therapy. Also provided, are pharmaceutical compositions comprising the HPβCD, optionally together with additional chemotherapeutic or cancer therapy agents.

### BACKGROUND OF THE INVENTION

The present invention relates to a compound, 2-hydroxypropyl-β-cyclodextrin (HPβCD) for use in methods of treating cancer, as well as to methods of treating a cancer using the compound HPβCD and to uses of the compound in the manufacture of one or more medicaments for the treatment of cancer. Also provided, are pharmaceutical compositions comprising the HPβCD, and optionally additional chemotherapeutic or cancer therapy agents.

Cancer is one of the leading causes of deaths worldwide. According to WHO, the number of new cancer cases worldwide is expected to rise by about 70% over the next two decades. Breast cancer is one of the most commonly occurring malignancies in women around the world, with almost 1.7 million new cases identified in 2012. In 2012, over 8 000 new cases of breast cancer were diagnosed in South Africa, making up 21.79% of the total cancer diagnoses nationally (National Health Laboratory Service, 2012).

Current treatments of cancer include chemotherapy and the surgical removal of tumours where possible. Metastasis and relapse are primary causes of poor survival in patients, even after successful treatment. EMT is a cellular program used by tumour cells to develop metastatic and drug resistance properties. The treatments available for breast cancer include use of selective estrogen receptor modulator (SERM) drugs which include tamoxifen (a gold standard drug) which is known to have several side effects. These side effects include the development of uterine cancer, cataracts, blood clots and heart attacks. Despite significant progress in anti-cancer therapeutics, many cancer patients either possess or acquire resistance to drug therapy and often present with disease relapse. Importantly, the invasion and metastasis facilitated by malignant cancer cells account for 90% of cancer patient deaths. As a result, attempting to develop novel means of combating cancer metastasis and drug resistance is pivotal.

Emerging experimental evidence continues to highlight cholesterol dyshomeostasis as a pivotal process in facilitating cancer development and drug resistance. Additionally, tumour metastasis usually favours the conversion between epithelial and mesenchymal cellular states. Epithelial-Mesenchymal Transition (EMT) has been reported to contribute to drug resistance which allows for cancer recurrence and metastasis, following chemotherapy. The abundance of cholesterol, in addition to directly regulating protein intermediates, promotes an increase in the presence of lipid rafts which serve as key signalling hubs crucial for EMT induction. Cholesterol accumulates in specified regions of the cell membrane, together with sphingolipids, forming micro-domains termed lipid-rafts. Lipid-rafts are known to be central to key signalling pathways involved in survival, migration, cell cycle progression as well as tumour development and metastasis. When cells undergo EMT, the significant increase in the cholesterol content as well as a decrease in the ceramide pool is documented to enhance plasma membrane fluidity. Stabilisation of lipid rafts has been postulated as an emerging therapeutic target to reduce the EMT-mediated increase in metastatic potential. This is supported by studies documenting that cancer cells that have undergone EMT display increased sensitivity to cholesterol targeting agents namely statins which is seen to deplete the cholesterol content in cells. By reducing membrane fluidity, cell motility and metastatic potential are reduced.

Seeing that the induction of EMT is associated with increased drug resistant potential, it can be inferred that cholesterol through its EMT-modulatory effects promotes increased drug resistance potential. Additionally, deregulated cholesterol metabolism is also seen to contribute to drug resistance by conferring cells with chemo-resistant traits. This is achieved by cholesterol-mediated regulation of pro-survival and anti-apoptotic pathways. Additionally, based on the crucial role cholesterol plays in maintaining lipid raft integrity, evidence exists implicating lipid rafts as key domains for maintaining the activity of multi drug resistance transporters. This therefore points to the multifaceted role of cholesterol in promoting EMT and mediating cancer drug resistance.

Attempts made at targeting cellular cholesterol by employing cholesterol synthesis inhibition to restore sensitivity have been widely exploited in the laboratory setting. This has predominantly been achieved by employing the most widely used lipid lowering agents on the market, i.e., statins. While employing cholesterol synthesis inhibition to reduce cellular cholesterol levels and enhance drug sensitivity has proven effective in a laboratory setting, the debilitating side effects associated with statin therapy is seen as a major limitation in a clinical setting. Common side effects include and are not limited to: muscle pains, hepato- and renal-toxicity, neurocognitive decline, myopathy as well as idiopathic polyneuropathy. Importantly, 30% of the patient population display non-responsiveness to statin therapy. This could be attributed to the long-term impediment of cholesterol synthesis in cells where cholesterol plays an integral role in regulating essential cellular processes. Consequently, the discovery of new cholesterol lowering agents is pivotal to combat drug resistance.

The inventors of the present invention propose that targeting cellular cholesterol could be a promising area in the search for novel cancer therapeutics and have identified a compound that may be useful as an adjuvant or neoadjuvant therapy.

### SUMMARY OF THE INVENTION

The present invention relates to a compound, 2-hydroxypropyl-β-cyclodextrin (HPβCD) for use in a method of treating cancer, to methods of treating a cancer using the compound HPβCD and to uses of the compound in the manufacture of one or more medicaments for treating cancer. Further provided according to the invention, are pharmaceutical compositions comprising the HPβCD, and optionally additional chemotherapeutic agents.

According to a first aspect of the present invention there is provided 2-hydroxypropyl-β-cyclodextrin (HPβCD) for use in a method of treating cancer, the method comprising administering the HPβCD in conjunction or combination with another cancer therapy to a subject in need thereof.

In a first embodiment of the HPβCD for use, the HPβCD may be administered as a neoadjuvant prior to another cancer therapy.

According to a second embodiment of the HPβCD for use, when the HPβCD is administered as a neoadjuvant, administering the HPβCD improves the sensitivity of cells of the cancer to the cancer therapy by reversing an EMT phenotype of the cells of the cancer.

In a third embodiment of the HPβCD for use, administering the HPβCD as a neoadjuvant may improve the subject's response to the cancer therapy.

According to a fourth embodiment of the HPβCD for use, the HPβCD may be administered as an adjuvant together with, or following, another cancer therapy.

In a further embodiment of the HPβCD for use, administering the HPβCD as an adjuvant sensitizes non-proliferative or proliferative cancer cells to the cancer therapy.

According to a further embodiment of the HPβCD for use, administering the HPβCD may reduce the toxicity of the cancer therapy and/or reduce one or more side-effects of the cancer therapy. For example, the subject may display better tolerance to the cancer therapy, such as oxaliplatin cancer therapy or fluorouracil cancer therapy, when combined with HPβCD.

According to another embodiment of the HPβCD for use, administering the HPβCD as an adjuvant reduces the risk or incidence of relapse of the cancer.

In yet a further embodiment of the HPβCD for use, the HPβCD is administered in conjunction or combination with another cancer therapy selected from the group consisting of chemotherapy, radiation therapy, hormone therapy, targeted therapy, or biological therapy.

According to a second aspect of the present invention there is provided for a method of treating cancer, comprising administering 2-hydroxypropyl-β-cyclodextrin (HPβCD) in conjunction or combination with another cancer therapy to a subject in need thereof.

In a first embodiment of the method, the HPβCD may be administered as a neoadjuvant prior to another cancer therapy.

According to a second embodiment of the method, when the HPβCD is administered as a neoadjuvant, administering the HPβCD may improve the sensitivity of cells of the cancer to the cancer therapy by reversing an EMT phenotype of the cells of the cancer.

In a third embodiment of the method, administering the HPβCD as a neoadjuvant may improve the subject's response to the cancer therapy.

According to a fourth embodiment of the method, the HPβCD may be administered as an adjuvant together with, or following, another cancer therapy.

In a further embodiment of the method, administering the HPβCD as an adjuvant may sensitize non-proliferative or proliferative cancer cells to the cancer therapy.

According to another embodiment of the method, administering the HPβCD as an adjuvant reduces the risk or incidence of relapse of the cancer.

According to a further embodiment of the method, administering the HPβCD may reduce the toxicity of the cancer therapy and/or reduce one or more side-effects of the cancer therapy. For example, the subject may display better tolerance to the cancer therapy, such as oxaliplatin cancer therapy or fluorouracil cancer therapy, when combined with HPβCD.

In yet a further embodiment of the method, the HPβCD may be administered in conjunction or combination with another cancer therapy selected from the group consisting of chemotherapy, radiation therapy, hormone therapy, targeted therapy, or biological therapy.

According to a third aspect of the present invention there is provided for the use of 2-hydroxypropyl-β-cyclodextrin (HPβCD) in the manufacture of a medicament for use in a method of treating cancer, the method comprising administering the medicament in conjunction or combination with another cancer therapy to a subject in need thereof. It will be appreciated by those of skill in the art that, where the additional cancer therapy is a chemotherapeutic agent, or a biological agent, hormone, or targeted therapy agent, the medicament may be formulated together with such cancer therapy or separately therefrom.

In a first embodiment of the use, the medicament comprising the HPβCD may be administered as a neoadjuvant prior to another cancer therapy.

According to a second embodiment of the use, when the medicament comprising the HPβCD is administered as a neoadjuvant, administering the medicament may improve the sensitivity of cells of the cancer to the cancer therapy by reversing an EMT phenotype of the cells of the cancer.

In a third embodiment of the use, administering the medicament comprising the HPβCD as a neoadjuvant may improve the subject's response to the cancer therapy.

According to a fourth embodiment of the use, the medicament comprising the HPβCD may be administered as an adjuvant together with, or following, another cancer therapy.

In a further embodiment of the use, administering the medicament comprising the HPβCD as an adjuvant may sensitize non-proliferative or proliferative cancer cells to the cancer therapy.

According to a further embodiment of the use, administering the medicament comprising HPβCD may reduce the toxicity of the cancer therapy and/or reduce one or more side-effects of the cancer therapy. For example, the subject may display better tolerance to the cancer therapy, such as oxaliplatin cancer therapy or fluorouracil cancer therapy, when combined with the medicament comprising HPβCD.

According to another embodiment of the use, administering the medicament comprising the HPβCD as an adjuvant reduces the risk or incidence of relapse of the cancer.

In yet a further embodiment of the use, the medicament comprising the HPβCD may be administered in conjunction or combination with another cancer therapy selected from the group consisting of chemotherapy, radiation therapy, hormone therapy, targeted therapy, or biological therapy.

According to a fourth aspect of the present invention there is provided for a pharmaceutical composition comprising 2-hydroxypropyl-β-cyclodextrin (HPβCD) and a pharmaceutically acceptable carrier, optionally together with a chemotherapeutic agent. It will be appreciated by those of skill in the art that the pharmaceutical composition may be formulated together with the chemotherapeutic agent or separately therefrom.

In one embodiment, the pharmaceutical composition, may further comprise pharmaceutical excipients, diluents, carriers or other suitable additives.

### BRIEF DESCRIPTION OF THE FIGURES

Non-limiting embodiments of the invention will now be described by way of example only and with reference to the following figures:

FIG. 1: Effect of CoCl₂, TGF-β and IL-6 treatment on EMT Epithelial Marker E-cadherin. Protein expression of EMT molecular markers in control and CoCl₂, TGF-β and IL-6 treated cells was detected using immunofluorescence microscopy. E-cadherin expression is indicated by Alexa Fluor 488 staining (green) while nuclei are stained with DAPI (blue). The scale bar on each of the images represents 100 μM. An unpaired two-tailed t-test was employed for statistical analysis where data represent the mean±standard deviation. n=3. P<0.05, ** P<0.01, *** P<0.001 and ns=not significant.

FIG. 2: Effect of CoCl₂, TGF-β and IL-6 treatment on EMT Mesenchymal Marker Vimentin. Protein expression of EMT molecular markers in control and CoCl₂, TGF-β and IL-6 treated cells was detected using immunofluorescence microscopy. Vimentin expression is indicated by Texas Red staining (red) while nuclei are stained with DAPI (blue). The scale bar on each of the images represents 100 μM. An unpaired two-tailed t-test was employed for statistical analysis where data represent the mean±standard deviation. n=3. P<0.05, ** P<0.01, *** P<0.001 and ns=not significant.

FIG. 3: Quantifying E-cadherin expression EMT induced cells following treatment with cholesterol targeting agents. Visual Representation indicating the protein expression of E-cadherin following treatment with various cholesterol targeting agents (1 mM MβCD and 10 mM HPβCD) in: (a) MCF-7; (b) NMuMg; and (c) HT-29 cells, pre- and post-EMT induction. E-cadherin expression is indicated by Alexa Fluor 488 staining (green) while nuclei are stained with DAPI (blue). The scale bar on each of the images represents 100 μM. A one-way ANOVA as well as the Bonferroni post-hoc test was employed for statistical analysis where data represent the mean±standard deviation. n=3. * P<0.05, ** P<0.01, *** P<0.001 and ns=not significant.

FIG. 4: Quantifying vimentin expression in EMT induced cells following treatment with cholesterol targeting agents. Visual Representation indicating the protein expression of vimentin following treatment with various cholesterol targeting agents (1 mM MβCD and 10 mM HPβCD) in: (a) MCF-7; (b) NMuMg; and (c) HT-29 cells, pre- and post-EMT induction. Vimentin expression is indicated by Texas Red staining (red) while nuclei are stained with DAPI (blue). The scale bar on each of the images represents 100 μM. A one-way ANOVA as well as the Bonferroni post-hoc test was employed for statistical analysis where data represent the mean±standard deviation. n=3. * P<0.05, ** P<0.01, *** P<0.001 and ns=not significant.

FIG. 5: Assessing changes in EMT-related gene expression in response to cholesterol depletion in NMuMg cells. a) RT-qPCR analysis of transcriptional expression of CDH1, Vim, SNAI1, TWIST1, ZEB1, CTBP1, and SMAD4 genes in TGFβ-treated cells (post-EMT) relative to pre-EMT cells. b) RT-qPCR analysis of transcriptional expression of CDH1, Vim, SNAI1, TWIST1, ZEB1, CTBP1, and SMAD4 genes between untreated cells pre-EMT and post-EMT cells treated with 1 mM MβCD, 10 mM HPβCD, and 10 μM simvastatin for 2 hours. Relative expression is presented as log 2 fold changes between conditions. Data shown represents mean±standard deviation. Statistical significance was calculated using a two-tailed unpaired Student's t-test where * P<0.05, ** P<0.01, *** P<0.001, and ns indicates no significant change; n=3. c) Western blots comparing protein expression of E-cadherin, Vimentin, and N-cadherin pre- and post-EMT treated with 1 mM MβCD, 10 mM HPβCD, and 10 μM simvastatin for 2 hours with β-tubulin used as a loading control.

FIG. 6: Assessing the invasive potential of EMT-induced cells following treatment with cholesterol targeting agents. Visual Representation indicating the number of cells that have invaded following treatment with various cholesterol targeting agents (1 mM MβCD and 10 mM HPβCD) in: (a) MCF-7; (b) NMuMg; and (c) HT-29 cells, pre- and post-EMT induction. Nuclei staining with DAPI (blue) facilitates visualization. The scale bar on each of the images represents 100 μM. A one-way ANOVA as well as a Bonferroni post-hoc test was employed for statistical analysis where data represent the mean±standard deviation. n=3* P<0.05, ** P<0.01, *** P<0.001 and ns=not significant.

FIG. 7: Assessing Changes in cholesterol-related gene expression in response to cholesterol depletion in NMuMG cells. a) RT-qPCR analysis of transcriptional expression of ABCA1, ABCG1, SREBF2, LXR, LDLR, HMGCR, PCSK9, and LCAT genes in TGFβ-treated cells (post-EMT) relative to pre-EMT cells. b) RT-qPCR analysis of transcriptional expression of ABCA1, ABCG1, SREBF2, LXR, LDLR, HMGCR, PCSK9, and LCAT genes between untreated cells pre-EMT and post-EMT cells treated with 1 mM MβCD, 10 mM HPβCD, and 10 μM simvastatin for 2 hours. Relative expression is presented as log 2 fold changes between conditions. Data represents mean±standard deviation. Statistical significance was calculated using a two-tailed unpaired Student's t-test where * P<0.05, ** P<0.01, *** P<0.001, and ns indicates no significant change; n=3. c) Western blots comparing protein expression of ABCG1, HMGCR, LXR, SREBP1, and SREBP2 (N: nuclear and C: cytoplasmic) pre- and post-EMT treated with 1 mM MβCD, 10 mM HPβCD, and 10 μM simvastatin for 2 hours with β-tubulin used as a loading control.

FIG. 8: Evaluating the effect of targeting cholesterol on the functionality of the MDR1 transporter. a) Intracellular retention of fluorescent calcein in: (a) MCF-7; and (b) HT-29 cells was significantly increased following treatment with cholesterol depleting agents post-EMT. b) Data of HT-29 cells showed cholesterol depleting agents (1 mM MβCD and 10 mM HPβCD) to increase the intracellular retention of fluorescent calcein in cells treated with the FOLFOX chemotherapy cocktail. A one-way ANOVA as well as a Bonferroni post-hoc test was employed for statistical analysis where data represent the mean±standard deviation. n=3* P<0.05, ** P<0.01, *** P<0.001 and ns=not significant.

FIG. 9: Investigating the effect of HPβCD treated together with CRC chemotherapies on tumour size in vivo. NOD/SCID mice were treated with single treatments of 3 000 mg/kg of HPβCD, 10 mg/kg of OXAL and 30 mg/kg of 5FU, as well as combinational therapies consisting of HPβCD+OXAL and HPβCD+5FU. Visually tumour sizes were reduced post HPβCD treatment and further reduction was observed when combination therapies were administered.

FIG. 10: Graphical representation of mice tumour size and weight following combination treatments with HPβCD. Tumour size (top panel) of untreated NOD/SCID mice versus treated mice (3 000 mg/kg of HPβCD, 10 mg/kg of OXAL and 30 mg/kg of 5FU). Tumour weight (bottom panel) of untreated mice versus treated mice. A significant reduction in tumour size and tumour weight was obtained using combination treatments of HPβCD+OXAL or HPβCD+5FU compared to when these chemotherapies were administered alone. A one-way ANOVA as well as the Dunnett's post-hoc test was employed for statistical analysis where data represent the mean±standard deviation. n=4. * P<0.05, ** P<0.01, *** P<0.001 and ns=not significant.

FIG. 11: Graphical representation showing average weekly weight of mice over a period of 15 weeks.

FIG. 12: Graphical representation of NOD/SCID mice serum cholesterol levels post treatment. Mice treated with HPβCD alone displayed a slightly higher average level of serum cholesterol (65.5 mg/dL) as compared to the average of untreated (51 mg/dL) mice. A one-way ANOVA as well as the Dunnett's post-hoc test was employed for statistical analysis where data represent the mean±standard deviation. n=4. There was no significant difference in serum cholesterol between all treatment groups.

FIG. 13: Graphs of AST and ALT, as a measure of liver toxicity in NOD/SCID mice post treatment. ALT measurements are similar across the untreated group and all the treatment groups (top panel). Ratios of AST:ALT (U/L) of greater than 2 indicate liver toxicity, and this was only observed in the HPβCD+OXAL treatment (bottom panel). A one-way ANOVA as well as the Dunnett's post-hoc test was employed for statistical analysis where data represent the mean±standard deviation. n=4. There was no significant difference in toxicity between all treatment groups.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The inventors of the present invention have identified the role of cholesterol depletor HPβCD in post-EMT cellular state and have found that it has the ability to reverse the mesenchymal phenotype to epithelial phenotype. They have tested this concept in three cell lines, using three different EMT induction methods and have found similar results in all cell lines. This demonstrates that HPβCD has the capability to reverse mesenchymal to epithelial phenotype in different cancer types and irrespective of the EMT induction mediator.

Based on the experimental findings described herein, the inventors have shown that targeting cellular cholesterol, through cholesterol depletion, combats EMT, decreases the invasive and metastatic potential of cells and also restores sensitivity to conventional chemotherapeutic agents. The inventors have further shown, through in vivo studies in mice, that administering the HPβCD reduces the toxicity of the cancer therapy and may thus be used to reduce one or more side-effects of the cancer therapy, such as oxaliplatin cancer therapy or fluorouracil cancer therapy, while preventing metastasis.

The inventors thus propose that combining HPβCD with neoadjuvant and adjuvant treatment using conventional cancer therapies may improve patient response to treatment, as well as reduce relapse rate. Additionally, the reduced drug resistance observed with HPβCD treatment may allow for lower doses of chemotherapeutic agents, lowering the harsh side effects usually associated with high chemotherapy doses.

As set out above, HPβCD has potential in neoadjuvant and an adjuvant therapy. Neoadjuvant therapy includes chemotherapy, radiation, or hormone therapy, prescribed before surgical removal of the tumour. Combining HPβCD with current neoadjuvant therapies could enhance their effect by improving drug sensitivity in the tumour by reversing the EMT phenotype.

As adjuvant therapy, HPβCD, combined with current therapies, could lower the risk of relapse. Current treatments, such as chemotherapy and radiation therapy target proliferative cells. Relapse occurs when non-proliferative tumour cells remain after treatment. Adding HPβCD to the adjuvant therapy regimen may sensitize both non-proliferative and/or proliferative cells to currently available treatment, reducing the risk of relapse.

It is thus proposed that HPβCD can work well in combination with current chemotherapeutic drugs to reduce their toxicity, improve efficacy, prevent metastasis and reduce drug resistance of tumour. HPβCD would also have potential benefits as long-term therapy to prevent recurrence or spread of cancer. As set out in the Examples below, HPβCD has the potential to reverse the mesenchymal phenotype back to epithelial phenotype, therefore, it demonstrates that HPβCD has the ability to prevent spread to the cancer.

The pharmaceutical compositions and compounds of the invention can be provided either alone or in combination with other compounds (for example, nucleic acid molecules, small molecules, peptides, or peptide analogues), in the presence of a liposome, an adjuvant, or any carrier, such as a pharmaceutically acceptable carrier and in a form suitable for administration to mammals, for example, humans.

As used herein a “pharmaceutically acceptable carrier” or “excipient” includes any and all antibacterial and antifungal agents, coatings, dispersion media, solvents, isotonic and absorption delaying agents, and the like that are physiologically compatible. A “pharmaceutically acceptable carrier” may include a solid or liquid filler, diluent or encapsulating substance which may be safely used for the administration of the pharmaceutical compositions or compounds to a subject. The pharmaceutically acceptable carrier can be suitable for intramuscular, intradermal, intravenous, intraperitoneal, subcutaneous, oral or sublingual administration. Pharmaceutically acceptable carriers include sterile aqueous solutions, dispersions and sterile powders for the preparation of sterile solutions. The use of media and agents for the preparation of pharmaceutically active substances is well known in the art. Where any conventional media or agent is incompatible with the active compound, use thereof in the pharmaceutical compositions of the invention is not contemplated. Supplementary active compounds can also be incorporated into the compositions.

Suitable formulations or compositions to administer the pharmaceutical compositions and compounds of the present invention to subjects fall within the scope of the invention. Any appropriate route of administration may be employed, such as, parenteral, intravenous, subcutaneous, intramuscular, intracranial, intraorbital, ophthalmic, intraventricular, intracapsular, intraspinal, intrathecal, intracistemal, intraperitoneal, intranasal, aerosol, topical, or oral administration.

The invention also relates in part to a method of providing an adjuvant or neoadjuvant therapy in combination with another cancer therapy, for treating cancer in a subject in need thereof, comprising administering to a subject in need thereof a therapeutically effective amount, of the compounds, compositions or formulations thereof of the present invention, in order to treat cancer in the subject.

Typically, an effective amount of the compounds or compositions of the invention will be administered to a subject. As used herein the term “subject” includes mammals, preferably human or animal subjects, but most preferably the subjects are human subjects. For pharmaceutical compositions, an effective amount of the compounds of the present invention can be provided, either alone or in combination with other compounds, or they may be linked with suitable carriers and/or other molecules, such as lipids.

In some embodiments, the pharmaceutical compositions or compounds according to the invention may be provided in a kit, optionally with a carrier, together with instructions for use.

An “effective amount” of a compound or pharmaceutical composition according to the invention includes a therapeutically effective amount. A “therapeutically effective amount” refers to an amount effective, at dosages and for periods of time necessary, to achieve the desired therapeutic result, such as treatment of cancer. The outcome of the treatment may for example be the prevention of cancer metastasis, preventing recurrence of the cancer, a decrease in cancer markers, a decrease in tumour size, inhibition of target metabolic pathways, delay in development of a pathology associated with cancer, or any other method of determining a therapeutic benefit. A therapeutically effective amount of a compound may vary according to factors such as the disease state, age, sex, and weight of the individual, the ability of the compound to elicit a desired response in the individual, previous therapeutic treatments, the nature and severity of the cancer to be treated, the route of administration, and the form of the composition. Dosage regimens may be adjusted to provide the optimum therapeutic response. A therapeutically effective amount is also one in which any toxic or detrimental effects of the compound are outweighed by the therapeutically beneficial effects.

The compounds or compositions of the present invention are intended for use as an adjuvant therapy or neoadjuvant therapy. As referred to herein, the term “adjuvant therapy” refers to an additional cancer treatment given together with, or after the primary treatment to lower the risk that the cancer will come back or recur. The primary therapy may include chemotherapy, radiation therapy, hormone therapy, targeted therapy, or biological therapy. As referred to herein, the term “neoadjuvant therapy” refers to treatment given as a first step to shrink a tumour or decrease a cancer before the main treatment, usually surgery, is given.

Examples of neoadjuvant therapy include chemotherapy, radiation therapy, and hormone therapy. Thus, the compounds and compositions of the present invention are intended for use together with other cancer therapies, including chemotherapy, radiation therapy, hormone therapy, targeted therapy, or biological therapy.

The amount of 2-hydroxypropyl-beta-cyclodextrin (HPβCD) in the composition may vary according to factors such as the cancer stage, age, sex, and weight of the individual. Dosage regimens may be adjusted to provide the optimum therapeutic response. For example, a single dose may be administered, or multiple doses may be administered over time. It may be advantageous to formulate the compositions in dosage unit forms for ease of administration and uniformity of dosage. The dosages of the compositions of the invention may be readily determined by techniques known to those of skill in the art or as taught herein.

Dosage values may vary and be adjusted over time according to the individual need and the judgment of the person administering or supervising the administration of the pharmaceutical compositions or compounds of the invention. It may be advantageous to formulate the compositions in dosage unit forms for ease of administration and uniformity of dosage.

The compounds and compositions of the present invention are intended to treat a cancer, to prevent metastasis of a cancer, or to prevent relapse of a cancer.

The term “preventing”, when used in relation to a medical disease or condition, is well understood in the art, and includes administration of a composition which reduces the frequency of or delays the onset of symptoms of a condition in a subject relative to a subject which does not receive the composition.

The term “therapeutic” treatment is well known to those of skill in the art and includes administration to a subject of one or more of the pharmaceutical compositions or compounds of the invention. If the composition is administered after manifestation of the unwanted condition, the treatment is therapeutic (i.e., it is intended to diminish, ameliorate, or stabilise the existing unwanted condition or side effects thereof).

Toxicity and therapeutic efficacy of compositions of the invention may be determined by standard pharmaceutical procedures in cell culture or using experimental animals, such as by determining the LD₅₀ and the ED₅₀. Data obtained from the cell cultures and/or animal studies may be used to formulate a dosage range for use in a subject. The dosage of any composition of the invention lies preferably within a range of circulating concentrations that include the ED₅₀ but which has little or no toxicity. The dosage may vary within this range depending upon the dosage form employed and the route of administration utilised. For compounds and compositions of the present invention, the therapeutically effective dose may be estimated initially from cell culture assays.

The following examples are offered by way of illustration and not by way of limitation.

### Example 1

Three different mechanisms of action were used to induce EMT in breast and colorectal adenocarcinoma cell lines. Breast cancer MCF-7 cells were treated with 150 micromolar (μM) cobalt chloride (CoCl₂) for 48 hours. Initially, a working stock solution of 2500 μM COCl₂ was prepared by dilution in distilled water. The resulting solution was further diluted upon administration to cells cultured in cell culture media. Cell culture media composition: Dulbecco's Modified Eagle's Medium (DMEM) (Sigma Aldrich, UK) supplemented with 10% foetal bovine serum (FBS) (Celtic Molecular Diagnostics, SA) and 1% Penicillin-Streptomycin (Sigma Aldrich, UK). NMuMg cells (a normal mouse mammary cell line) were treated with 10 nanograms/millilitre of transforming growth factor beta 1 (TGF-β) for 48 hours. A stock solution of TGF-β was prepared by reconstituting 2 μg of recombinant TGF-β at 20 μg/mL in sterile 4 mM HCl containing 1 mg/mL of bovine serum albumin (BSA) diluted in water. NMuMG cells were cultured in Dulbecco's Modified Eagle Medium (DMEM) (ThermoFisher Scientific, USA), supplemented with 10% FBS, 1% Penicillin-Streptomycin, 1% GlutaMAX, and 10 μg/mL insulin. Colorectal cancer HT-29 cells were treated with 50 nanograms/millilitre of interleukin-6 (IL-6) for 24 hours. A working stock solution of 1 micrograms/millilitre of IL-6 was prepared by dilution in 0.1% BSA in phosphate-buffered saline (PBS). The resulting solution was further diluted upon administration to cells cultured in cell culture media. Cell culture media composition: McCoy's 5a (Modified) Medium (Sigma Aldrich, UK), supplemented with 10% FBS and 1% Penicillin-Streptomycin.

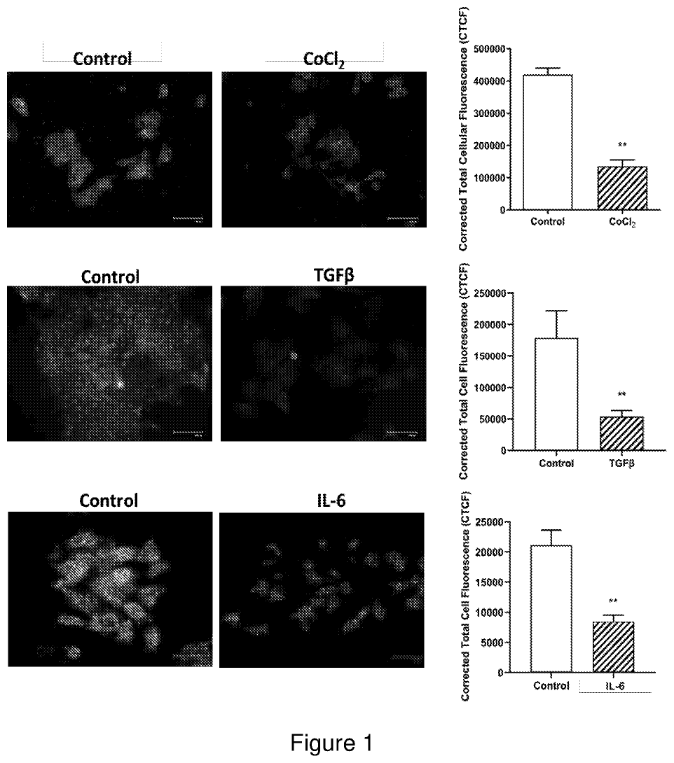

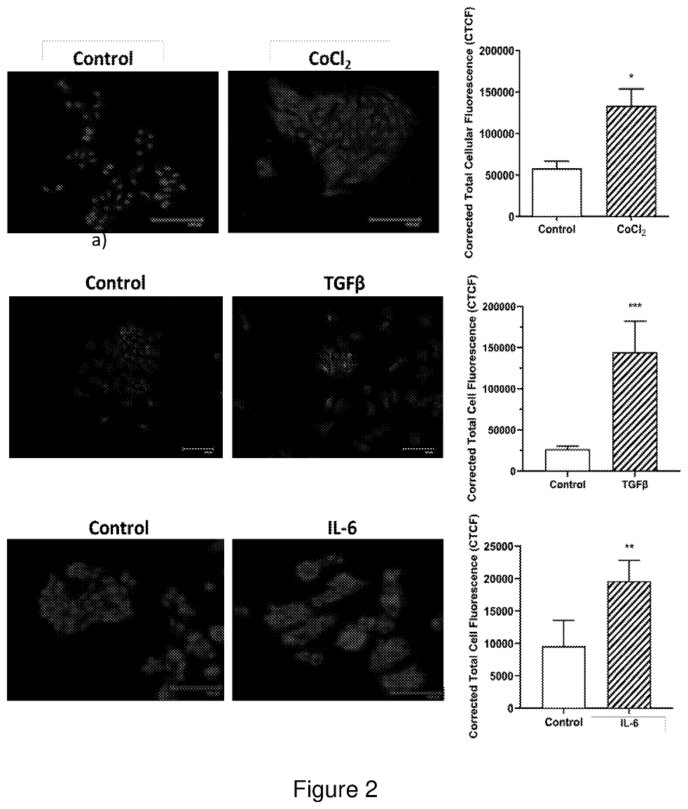

Protein expression of EMT molecular markers in control and CoCl₂, TGF-β and IL-6 treated cells was detected by employing immunofluorescence microscopy. The epithelial cell junction protein E-cadherin is used as a molecular marker to indicate cells that possess an epithelial phenotype, whereas the intermediate filament vimentin is used as a molecular marker to indicate cells that possess a mesenchymal phenotype. E-cadherin expression was detected by Alexa Fluor 488 staining (green) while nuclei were stained with DAPI (blue) (FIG. 1). Vimentin expression was detected by Texas Red staining (red) while nuclei were stained with DAPI (blue) (FIG. 2). Images were captured using the Floid™ Cell Imaging System followed by analysis using the Image J software.

From FIGS. 1 and 2, it is evident that E-cadherin expression is significantly decreased following EMT induction with CoCl₂, TGF-β and IL-6 and that Vimentin expression is significantly increased following EMT induction with CoCl₂, TGF-β and IL-6. Thus, all treatment conditions led to a significant decrease in the expression of E-cadherin (Epithelial marker) and increased expression of Vimentin (mesenchymal marker).

### Example 2

Findings from laboratory studies showed that following successful EMT induction of breast (MCF-7 and NMuMg) and colorectal cancer (HT-29) cells, cholesterol depletion with compound HPβCD showed an increase in epithelial marker E-cadherin and decrease in mesenchymal marker vimentin as indicated by immunofluorescence staining. Methyl-β-cyclodextrin (MβCD) was employed as a positive control, as it is a well-documented cholesterol depletory agent. Following the 2-hour treatment periods, cells were fixed (4% formaldehyde—10 minutes), permeabilized (0.1% Triton-X-100, 10 minutes) and a blocking step completed (1% Bovine Serum Albumin made up in 0.1% Tween-20-1 hour). Cells were incubated at 4° C. in primary antibody overnight (1:250 dilution) washed with phosphate-buffered saline (PBS) and subsequently incubated in secondary antibody (1:1000 dilution) for 45 minutes at room temperature. Following nuclear counter-staining, coverslips containing attached cells were mounted and cells were subsequently visualised by using the Floid™ Cell Imaging System (ThermoFisher Scientific, USA). Immunofluorescence intensity was quantified by utilising the ImageJ software (NIH, USA). Cells of interest and background fluorescence was analysed by considering the following parameters: area, integrated density and mean grey value. Corrected total cellular fluorescence was calculated as follows: CTCF=Integrated Density−(Area of selected cell×Mean fluorescence of background readings).

The protein expression of E-cadherin following treatment with various cholesterol targeting agents in MCF-7, NMuMg, and HT-29 cells, pre- and post-EMT induction was investigated. E-cadherin expression was determined by Alexa Fluor 488 staining (green) while nuclei were stained with DAPI (blue).

The protein expression of vimentin following treatment with various cholesterol targeting agents in MCF-7, NMuMg, and HT-29 cells, pre- and post-EMT induction was also investigated. Vimentin expression was determined by Texas Red staining (red) while nuclei were stained with DAPI (blue). Images were captured using the Floid™ Cell Imaging System followed by analysis using the Image J software. These results were further validated in the NMuMg cell line by analysing E-cadherin, Vimentin and N-cadherin expression using whole protein lysates.

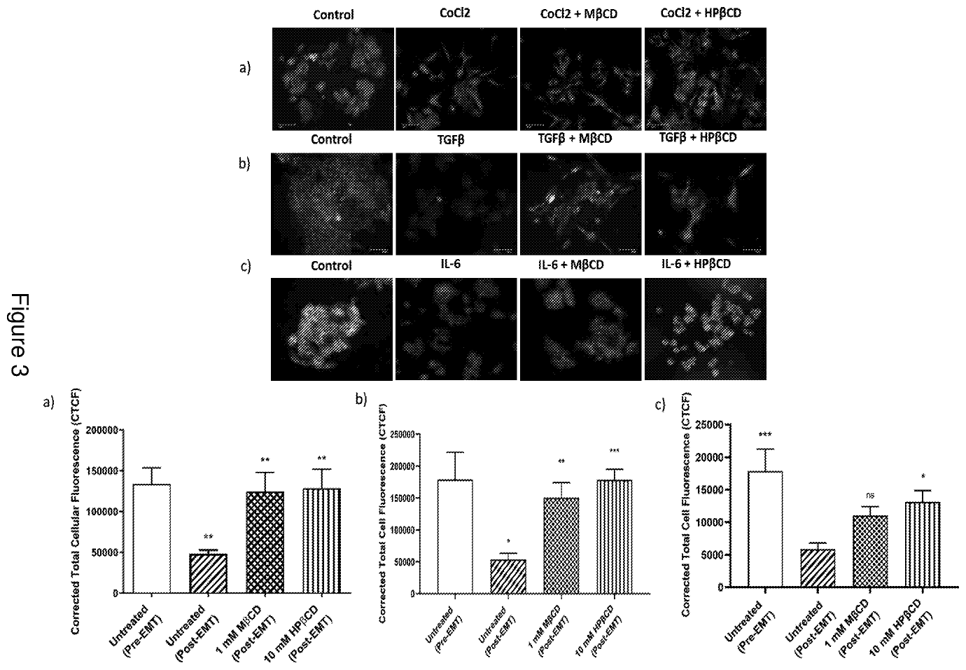

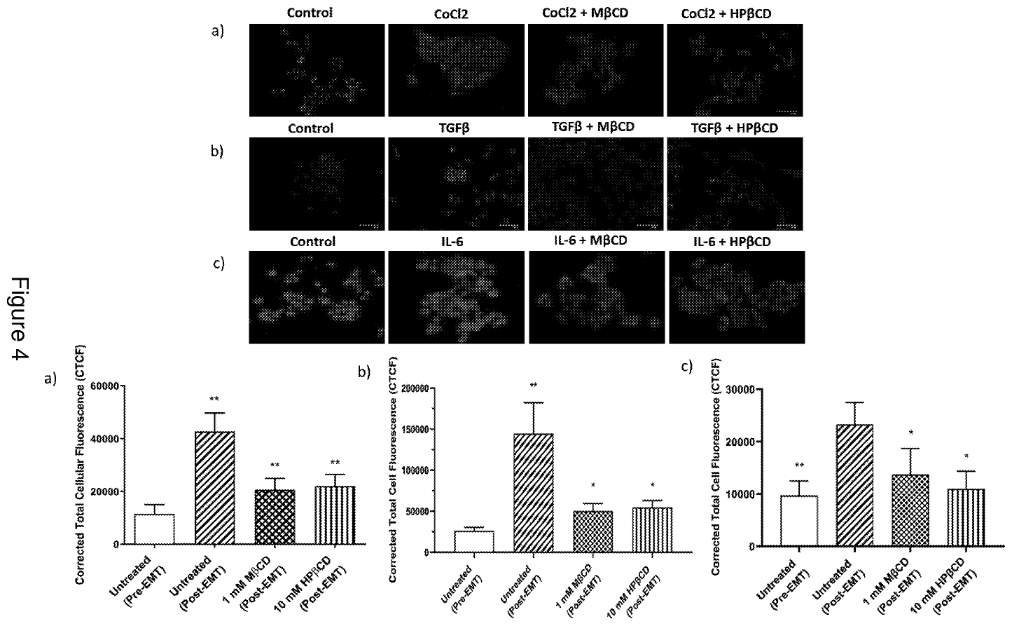

Based on these results, it is evident that treatment with cholesterol targeting agents leads to an increase in the expression of E-cadherin with the effect being significant in post-EMT cells (FIG. 3). Further, it is evident that treatment with cholesterol targeting agents leads to a decrease in the expression of vimentin with the effect being significant in post-EMT cells (FIG. 4).

These results corroborate well with the western blotting data obtained in the NMuMg cell line. Cells were harvested and lysed using 100 μl of lysis buffer per 10⁶ cells. Bicinchoninic acid (BCA) assay (Sigma Aldrich, UK) was performed to quantify the protein lysates, which were normalised against a BSA (bovine serum albumin (BSA) (Inqaba Biotec™, SA) standard curve. A final protein concentration of 10 mg/ml lysate was loaded into a 10% SDS-PAGE gel and transferred onto a PVDF membrane (0.22 μm pore size) using a semi-dry transfer system: Trans-Blot® Turbo™ Transfer System (Bio-Rad, USA). The membrane was then blocked using 3% BSA for 1-hour. Primary antibodies were added and incubated overnight, and the following day secondary antibodies were then added to the blots for 1-hour. The blots were viewed on a the ChemiDoc Imaging System (Bio-Rad, USA) using the Clarity™ Western ECL HRP substrate (Bio-Rad, USA).

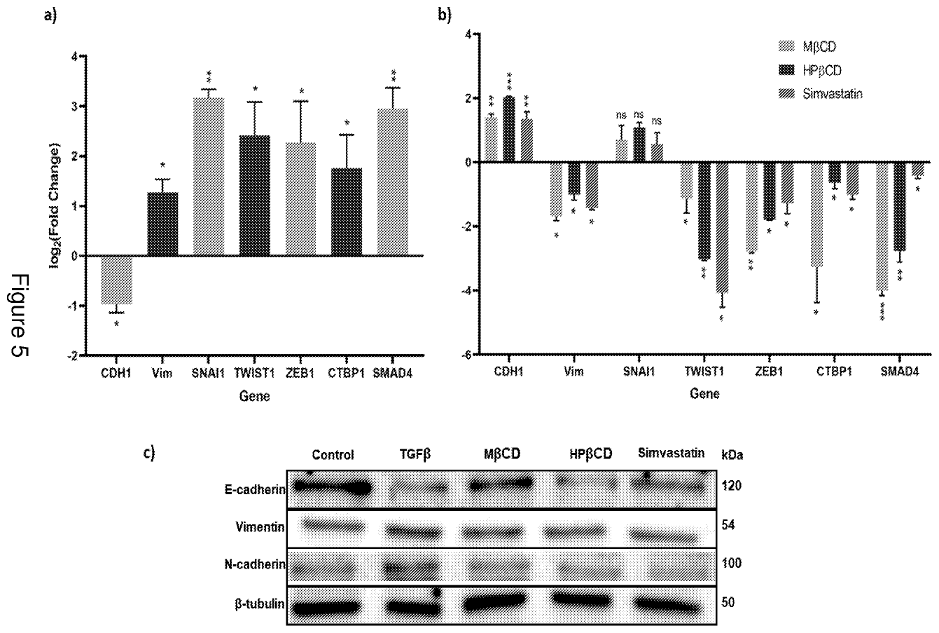

An epithelial marker, E-cadherin, and mesenchymal markers Vimentin and N-cadherin were analysed indicating decreased expression following treatment with cholesterol targeting agents (FIG. 5). Furthermore, treatment of the NMuMg cell line with cholesterol targeting agents resulted in a significant decrease in CTBP1 (major regulator) and SMAD4 (effector) which play a crucial role in TGF-β signalling. This result supports the observed decrease in the expression of EMT-related transcription factors TWIST1 and ZEB1 following treatment. It can thus be postulated that targeting cellular cholesterol in post-EMT cells helps promote restoration of an epithelial phenotype.

### Example 3

The number of cells that have invaded following treatment with various cholesterol targeting agents in MCF-7, NMuMG and HT-29 cells pre- and post-EMT induction was investigated and nuclei staining with DAPI (blue) was employed to facilitate visualization. To achieve this, cells were trypsinised following treatments and re-suspended in serum free media and subsequently seeded in a Geltrex-coated transwell chambers containing 0.8 μm pore size. The transwell chambers were placed into 24-well plates and incubated for 24-hours at 37° C. Following this incubation, non-invasive cells were removed from the top chamber with cotton swabs and the invaded cells were fixed (4% formaldehyde—10 minutes) and stained with DAPI. Images were captured using the Floid™ Cell Imaging System followed by analysis using the Image J software.

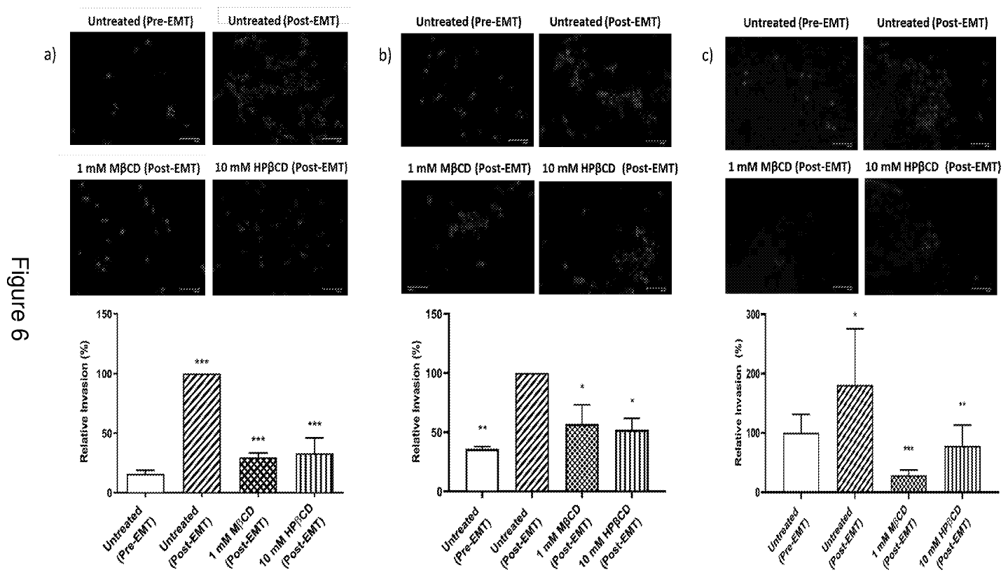

It is evident from the results in FIG. 6 that EMT leads to a significant increase in invasive potential of MCF-7, NMuMg, and HT-29 cells. Treatment with cholesterol targeting agents lead to a significant decrease in the invasive potential of post-EMT cells and could prove effective in reducing the metastatic potential of cancer cells. This can be attributed to the disruption in lipid raft integrity which facilitates the shedding of key cell surface receptor that cancer cells depend on for conferring an aggressive disease phenotype. Furthermore, several EMT related pathways are also inhibited. This facilitates an increased expression of key epithelial markers while reducing the expression of mesenchymal markers. As a result of this, employing cholesterol targeting agents effectively combats EMT and metastasis and prevents the acquisition of an aggressive disease phenotype.

To delineate the molecular mechanism governing cholesterol-mediated EMT induction, RT-qPCR analysis of the major cholesterol-related genes were assessed in the NMuMg cell line. RNA was extracted using the Direct-Zol™ RNA MiniPrep kit (Zymo Research, Inqaba Biotec™, SA). RNA extraction was carried out as per the manufacturer's protocol. Total RNA of 1 μg was utilised for cDNA synthesis using the RevertAidFirst Strand cDNA Synthesis kit (Thermo Fisher Scientific, USA) and was carried out according to the manufacturer's protocol. qPCR was performed using the SensiFAST™ SYBR® No-ROX kit, utilising a 3-step cycle on the CFX96 Touch™ Real-Time PCR detection system (Bio-Rad).

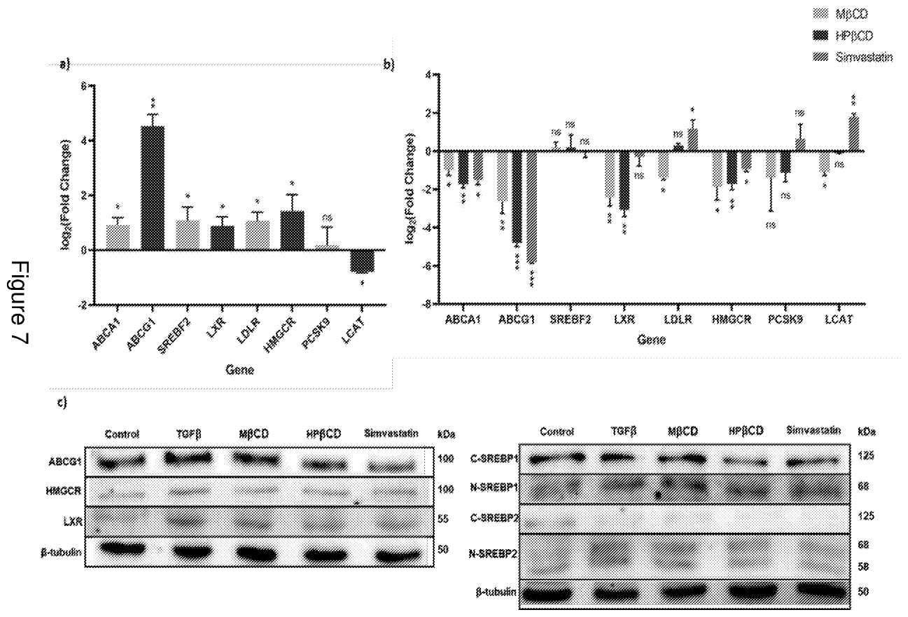

An increase in the expression of the master regulator LXR governing cholesterol efflux was documented supporting the observed decreased expression of major efflux genes ABCA1 and ABCG1 (FIG. 7). Moreover, the expression of the master regulator SREBF2 increased validating the increased expression of HMGCR, LDLR and PCSK9 in post-EMT cells. The observed effect was reversed following treatment with cholesterol targeting agents, with MβCD displaying a more significant effect on cholesterol biosynthesis and influx. Conversely, HPβCD and Simvastatin drastically affected cholesterol efflux.

To assess the multi-drug resistant potential of cells pre- and post-EMT, the Vybrant® MDR Assay Kit (ThermoFisher Scientific, USA) was employed. This assay relies on the administration of an appropriate multidrug resistance protein 1 (MDR1) substrate, calcein acetoxymethyl ester (calcein AM) where drug-efflux potential is assessed by measuring the levels of intracellular calcein fluorescence. 10 000 cells were seeded per well in a 96-black walled plate. Following the 2-hour treatment period, cells were incubated with 0.25 μM Calcein-AM for 30 minutes at 37° C. Following this, two PBS washes were completed, and PBS administered to each well to ascertain fluorescence intensity. The Victor Nivo multi-mode microplate reader (Perkin Elmer, USA) was utilised with the excitation maximum set to 494 nm and the emission maximum set to 517 nm.

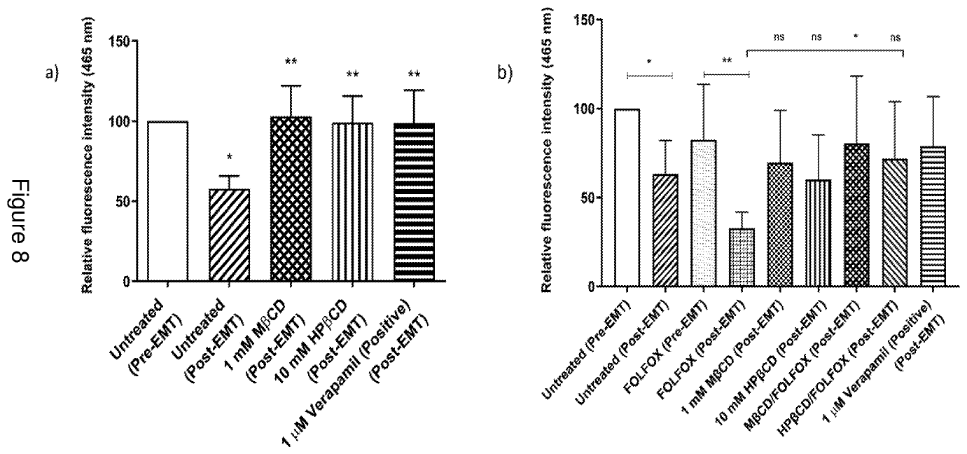

Intracellular retention of fluorescent calcein in MCF-7 and HT-29 cells was significantly increased following treatment with cholesterol depleting agents post-EMT, therefore potentiating treatment with cholesterol targeting agents as suitable means to abrogate MDR potential (FIG. 8). This can be attributed to alterations in lipid-raft membrane structure resulting in translocation of MDR1 to detergent soluble fractions which affects basal ATPase activity. This consequently affects the efflux potential of the MDR1 transporter. Preliminary data of HT-29 cells showed cholesterol depleting agents to increase the intracellular retention of fluorescent calcein in cells treated with the FOLFOX chemotherapy cocktail. Fluorescence intensity (RFU) was quantified using the Victor Nivo multi-mode microplate reader (Perkin Elmer, USA) with the excitation maximum set to 494 nm and the emission maximum set to 517 nm.

Taken together, these results show that employing cholesterol depletion also significantly decreased the invasive and drug resistant potential of cells. The experimental evidence provided above shows a reduction in the EMT phenotype after cholesterol depletion with HPβCD. With this reversal of EMT, the inventors have shown a reduction in invasive potential and drug resistance. They have also shown low toxicity with the use of HPβCD, compared to simvastatin (a cholesterol synthesis blocking statin).

### Example 4

In order to investigate the in vivo application of HPβCD as an anti-cancer agent for colorectal cancer (CRC) using NOD/SCID mice, 24 NOD/SCID mice (5-8 weeks old) were injected with 4×10⁶ HT-29 cells (suspended in 10% McCoy's media and 1×PBS), until tumours developed. Once tumours reached a size of 150-200 mm³ (1-2 weeks), treatments (12 treatments in total for each group, however only up to 6 treatments for the OXAL treated group due to obvious toxicity) were given. Treatment groups consisted of 4 mice each and included a control group (injected with 1×PBS), a group treated with HPβCD, a group treated with Oxaliplatin (OXAL) only, a group treated with 5-Fluorouracil (5FU) only, a group treated with HPβCD and OXAL and lastly a group treated with HPβCD and 5FU. Mice were intraperitoneally injected with 3 000 mg/kg of HPβCD, 10 mg/kg of OXAL and 30 mg/kg of 5FU thrice a week for 4 weeks. For combination treatments HPβCD was administered with either 5FU or OXAL chemotherapy. Mice were weighed three times a week and averages were calculated to show per week weights. When mice were euthanized, serum was collected from whole blood samples and this was further used to test cholesterol levels, as well as Aspartate aminotransferase (AST) and Alanine aminotransferase (ALT) as a measure for liver toxicity. They were then perfused and all organs, including tumours, were harvested. All organs were snap frozen and stored at −80° C. Tumour samples were further stored in 10% neutral buffered formalin (4° C.) and RNA Later (−80° C.).

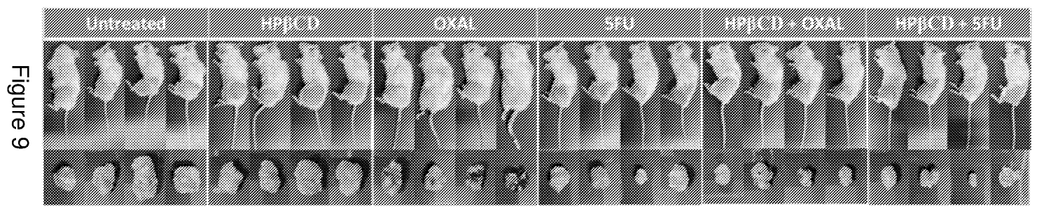

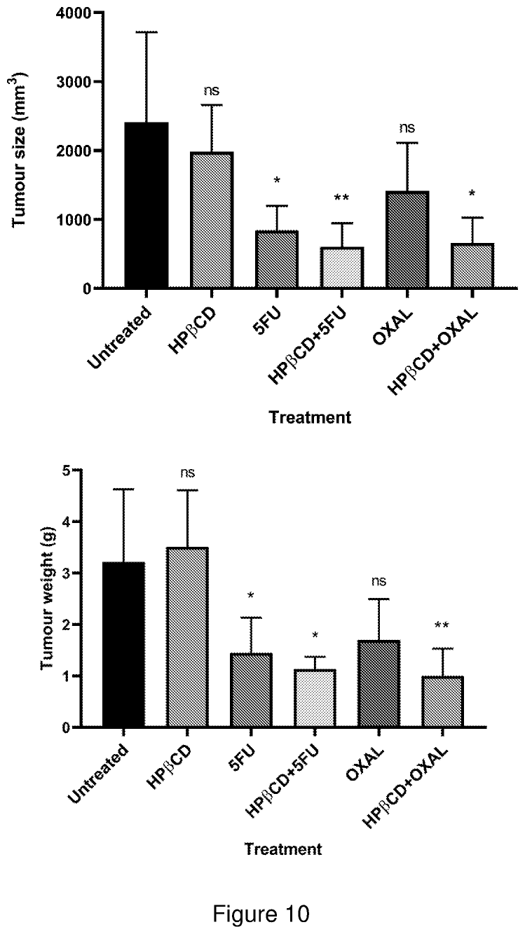

A significant reduction in tumour size and weight was observed. Visually, tumour sizes were reduced post HPβCD treatment and a further reduction was observed when combinational therapies were administered (FIG. 9). There was an approximate average of 17% reduction in tumour size in mice treated with HPβCD and an average of 31% further reduction in tumours treated with OXAL and HPβCD, as compared to OXAL treatment only. Additionally, HPβCD showed an overall ˜74% reduction in tumour size and weight was seen in both combinational therapies (Table 1 and FIG. 10). Thus, both combination treatments of HPβCD and OXAL or HPβCD and 5FU displayed a further reduction in tumour size as compared to when these chemotherapies were solely used. Therefore, it could be inferred that HPβCD increases the efficacy of these drugs. Interestingly, HPβCD decreased the toxicity of OXAL as the group treated with OXAL alone were only given 6 treatments, before falling ill (2 died and 2 had to be euthanized). While the group treated with OXAL and HPβCD could reach the end-point treatment (given all 12 treatments of the same dose) and were in good health. Post-death, dissection showed metastasis almost throughout the body in one mouse in OXAL only treated group, especially in liver.

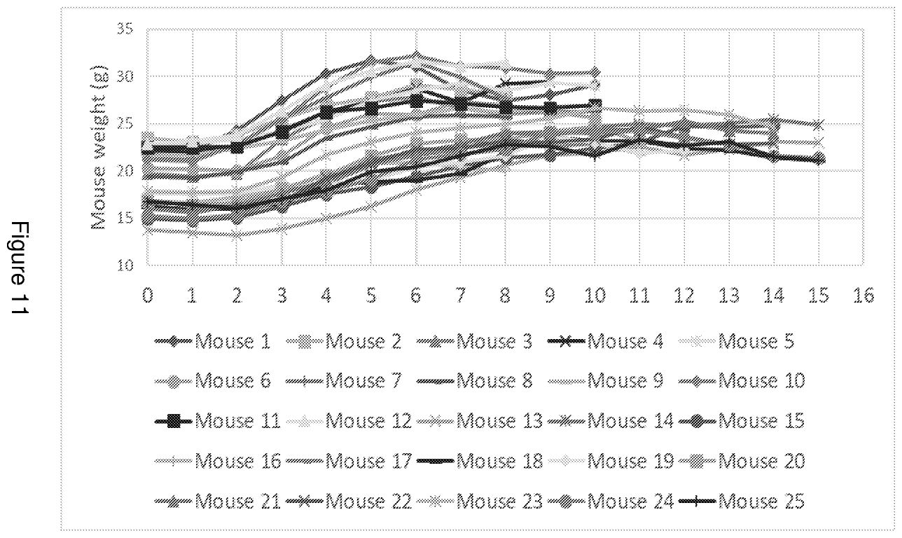

Mice were weighed thrice a week and average weekly weights were taken. Weights were monitored over the duration of the study as one of the indicators for mice welfare. The weekly weights of the mice are shown in FIG. 11.

TABLE 1
Tabulated results show tumour sizes and weight measured for the mice in each treatment
group, displaying tumour gain or reduction, as compared to the untreated group.

| | | Tumour | Tumour | Average | Average | Tumour | Tumour gain |
|---|---|---|---|---|---|---|---|
| | Mice | size | weight | tumour size | tumour weight | reduction | or reduction |
| Treatment | no. | (mm³) | (g) | (mm³) | (g) | (mm³) | (g) |
| Untreated | 2 | 932.598 | 1.9 | 2407.931839 | 3.2175 | — | — |
| | 5 | 2203.303032 | 2.54 | | | | |
| | 6 | 4116.103056 | 5.16 | | | | |
| | 9 | 2379.723267 | 3.27 | | | | |
| HPβCD | 1 | 1538.05392 | 2.79 | 1983.031038 | 3.5125 | −17.64588156 | 9.168609169 |
| | 3 | 1377.836388 | 2.54 | | | | |
| | 10 | 2871.975926 | 4.95 | | | | |
| | 11 | 2144.25792 | 3.77 | | | | |
| OXAL | 4 | 2419.346089 | 2.85 | 1413.352391 | 1.6975 | −41.30430237 | −47.24164724 |
| | 7 | 850.864392 | 1.04 | | | | |
| | 8 | 1320.614991 | 1.49 | | | | |
| | 12 | 1062.584094 | 1.41 | | | | |
| 5FU | 15 | 1095.66309 | 1.58 | 841.4406535 | 1.4475 | −65.05546212 | −55.01165501 |
| | 21 | 923.439352 | 1.68 | | | | |
| | 22 | 316.32264 | 0.47 | | | | |
| | 23 | 1030.337532 | 2.06 | | | | |
| HPβCD + | 17 | 710.234668 | 1 | 660.2862629 | 0.9975 | −72.57869794 | −68.997669 |
| OXAL | 18 | 1154.979288 | 1.75 | | | | |
| | 24 | 316.582083 | 0.66 | | | | |
| | 25 | 459.3490125 | 0.58 | | | | |
| HPβCD + | 13 | 721.895424 | 1.11 | 606.7502998 | 1.135 | −74.80201516 | −64.72416472 |
| 5FU | 14 | 718.011065 | 1.06 | | | | |
| | 19 | 109.3574405 | 0.91 | | | | |
| | 20 | 877.7372695 | 1.46 | | | | |

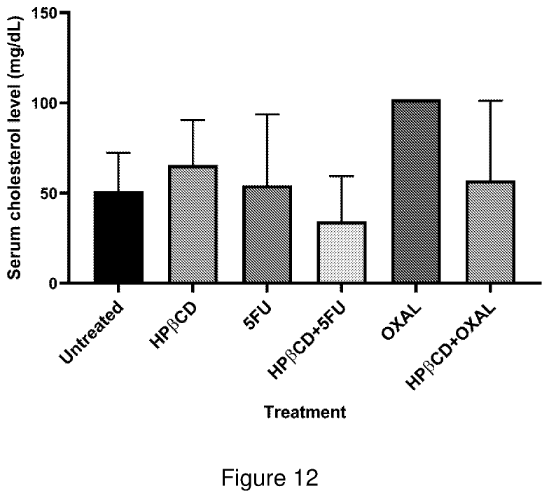

HPβCD treated mice displayed a slightly higher level of serum cholesterol (an average of 65.5 mg/dL) as compared to the untreated (an average of 51 mg/dL) mice (FIG. 12). This transient increase is expected due to cholesterol sequestration properties of HPβCD, allowing excess cholesterol to be effluxed into the blood. Interestingly, both OXAL and 5FU treated mice displayed significantly higher serum cholesterol levels. However, combination treatment with HPβCD was shown to have reduced serum cholesterol levels in the mice, which may lead to decreased uptake of cholesterol in these cancer cells and therefore, increasing the efficacy of OXAL and 5FU to reduce tumour growth. Another possible explanation can be that cyclodextrins are known excipients and increase the solubility of the drugs. On the contrary, OXAL and 5FU could possibly be increasing transport protein activity therefore enhancing drug efflux and possibly cholesterol simultaneously. However, combinational treatment with HPβCD reduced serum cholesterol levels. Cells could possibly be using more cholesterol, or the liver could be excreting cholesterol in the form of bile acids. The graphs show variation in the level of cholesterol in blood within a group of mice. For the OXAL treatment group, the inventors could only draw blood from one mouse as the others died due to toxicity of OXAL.

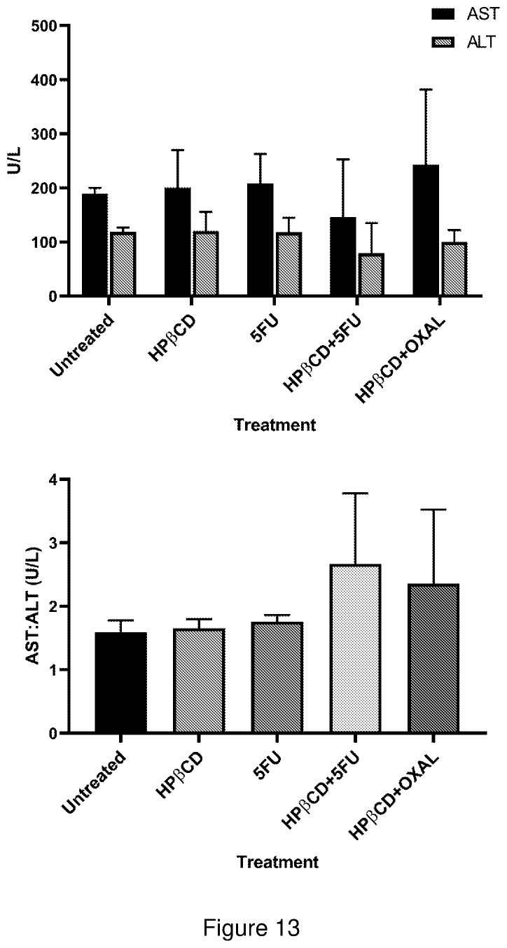

Blood was collected post euthanization and serum was analysed for AST and ALT levels using the IDEXX Catalyst DX Chemistry Analyzer. ALT is a more specific indicator for liver toxicity since there's a greater concentration in the liver compared to other tissues. FIG. 13 shows the levels of liver toxicity markers ALT and AST and their ratios. ALT measurements were similar across the untreated group and all the treatment groups (FIG. 13, top panel). There was no observed liver toxicity in the HPβCD group (an average of 1.67 U/L) compared to the untreated group (an average of 1.59 U/L). Ratios of greater than 2 for AST:ALT ratio indicate liver toxicity, and this was only observed in the HPβCD+OXAL treatment (FIG. 13, bottom panel). The AST:ALT ratio for the OXAL alone group could not be obtained. However, it is inferred that HPβCD reduced toxicity of OXAL alone. This is because 6 treatments of OXAL alone resulted in the unexpected death of 50% of the mice in this treatment group. As per ethical regulations, the remainder of the mice in the OXAL treatment group were euthanized. Due to the unexpected termination of this group, no suitable blood samples were obtained. Mice treated with OXAL in combination with HPβCD, could tolerate 12 doses of OXAL with no visible ill health effects.

Additionally, HPβCD was shown to reduce toxicity related to 5FU in mice. No metastasis was observed in combination treated groups with HPβCD. Liver toxicity was also not observed in most of the mice. A transient increase in cholesterol levels in HPβCD treated group shows that the treatment was working as HPβCD was extracting cholesterol from cells. Therefore, HPβCD can work well in combination with current chemotherapeutic drugs to reduce their toxicity, improve efficacy, prevent metastasis and reduce drug resistance of tumour.

1-9. (canceled)

10. A method of treating cancer, comprising administering 2-hydroxypropyl-β-cyclodextrin (HPβCD) in combination with another cancer therapy to a subject in need thereof.

11. The method of claim 10, wherein the HPβCD is administered as a neoadjuvant prior to another cancer therapy.

12. The method of claim 11, wherein administering the HPβCD improves the sensitivity of cells of the cancer to the cancer therapy by reversing an EMT phenotype of the cells of the cancer.

13. The method of claim 11, wherein administering the HPβCD improves the subject's response to the cancer therapy.

14. The method of claim 10, wherein the HPβCD is administered as an adjuvant together with, or following, another cancer therapy.

15. The method of claim 14, wherein administering the HPβCD sensitizes non-proliferative cancer cells to the cancer therapy.

16. The method of claim 14, wherein administering the HPβCD reduces the toxicity of the cancer therapy and/or reduces one or more side-effects of the cancer therapy.

17. The method of claim 14, wherein administering the HPβCD reduces the risk or incidence of relapse of the cancer.

18. The method of claim 10, wherein the cancer therapy is chemotherapy, radiation therapy, hormone therapy, targeted therapy, or biological therapy.

19-27. (canceled)

28. A pharmaceutical composition comprising 2-hydroxypropyl-β-cyclodextrin (HPβCD) and a pharmaceutically acceptable carrier, optionally together with a chemotherapeutic agent.

29. The pharmaceutical composition of claim 28, further comprising pharmaceutical excipients, diluents, carriers or other suitable additives.
